(19) United States
(12) Patent Application Publication
(10) Pub. No.: US 2018/0296363 A1
(43) Pub. Date: Oct. 18, 2018

(54) INTERBODY FUSION IMPLANT

(71) Applicant: Bret Michael Berry, Tallahassee, FL (US)

(72) Inventor: Bret Michael Berry, Tallahassee, FL (US)

(21) Appl. No.: 15/490,007

(22) Filed: Apr. 18, 2017

(51) Int. Cl. A61F 2/44; A61F 2/30

(52) U.S. Cl. CPC A61F 2/4465; A61F 2/30942; A61F 2002/30985; A61F 2002/3092; A61F 2002/4475

(57) ABSTRACT

The present invention generally relates to an interbody fusion implant. Specifically, the present invention relates to an implant that incorporates a purposefully designed three-dimensional matrix that optimizes bony ingrowth, with such matrix then being overmolded with a material that provides structural support.

### FIELD OF THE INVENTION

The present invention generally relates to an interbody fusion implant. Specifically, the present invention relates to an implant that incorporates a purposefully designed three-dimensional matrix that optimizes bony ingrowth, with such matrix then being overmolded with a material that provides structural support.

### BACKGROUND OF THE INVENTION

Interbody fusion (“IBF”) implants are commonly used in a variety of orthopedic applications. While the structure of an IBF implant is primarily based on application, a primary consideration for any IBF implant is achieving a design the maximizes strength, while also promoting bone growth to achieve proper fusion between the IBF implant and adjacent bone(s). Accordingly, IBF implant designs have incorporated both nonporous and porous structures, with the goal of having the nonporous areas providing strength and structural support to the implant and the porous areas promoting bone growth and fusion.

For example, a material known as trabecular metal is comprised of a lattice structure of tantalum metal. This material has been used for IBF implants and has demonstrated bone growth across the interbody space, which is evidence that a porous structure can aid in fusion throughout the implant. However, tantalum has a number of drawbacks including, that it is stiffer than bone, which leads to brittleness, and that it is highly radio-opaque, preventing post-operative examination of bone growth with imaging technology. Therefore, polyether ether ketone (“PEEK”) has become a popular material for IBF implants, as PEEK has an elastic modulus that is close to bone and is radiolucent.

While PEEK has a number of characteristics that are advantageous for use in IBF implants, there are also some drawbacks to using PEEK in such applications. The primary drawback is that PEEK is bio-inert, which can make fusion between the IBF implant and the bone more difficult. As a result, many methods have been tried to increase fusion in PEEK IBF implants including spraying on a porous titanium coating and making the PEEK itself porous. Each of these methods, however, suffers from additional limitations.

Generally, adding a coating to an IBF implant involves spraying an amorphous structure onto a solid substrate. While this method can achieve a combined nonporous, porous structure, it also has a number of drawbacks. First, the porous structure can only be a few millimeters thick because as more spray is added, the pores that were formed by earlier layers of spray become filled in by subsequent spray layers. Second, the spray can only be added to exterior surfaces of the IBF implant, which limits its advantages as the spray cannot reach center areas of the implant. For example, with a typical IBF implant, bone graft is placed into the center of the IBF implant and it is in the center of the implant that bone growth is desired. Third, even if the size of the spray media can be controlled, it is still a randomized process with randomized pore sizes and structures, making it difficult to create a consistent pore size and percent porosity. Finally, there is a high chance of the implant failing due to shear forces at the interface of the sprayed on porous structure and the underlying solid substrate.

Additionally, currently practiced methods of forming a porous structure from or within the PEEK material itself have a number of limitations, largely due to the resiliency and strength of PEEK. One method that has been attempted is to introduce gas bubbles into molten PEEK, thereby creating pores when the PEEK material sets. There are a number of problems with this gas-evaporative or bubble method. First, it is difficult to create a structure that is both porous and nonporous. Second, the porous structure created by the air bubble process has been shown to be inadequate for stimulating bony ingrowth. This is largely due to the fact that the porous structure created by the air bubbles does not maintain a consistent pore size, as the pores created by two or more gas variably sized bubbles touching one another. Finally, the air bubble process generally creates two spherical pores that are connected by a smaller transition pore. Studies have demonstrated that the spherical pores are too often large to promote adequate bone growth and fusion, while the transition pore is too small.

Another method that has been attempted is to sinter beads onto a solid substrate. With this method, beads of a certain material, for example PEEK, are melted or sintered so they stick together and form a porous structure. However, the beads must be forced together, to ensure the beads melt into one another; otherwise the structure can be weak. Furthermore, the sintering process creates a weak structure with small pores and low porosity percentage. Finally, the sintering method has been shown to result in IBF implants where beads of material break of the solid substrate, as there is a high chance of failure due to shear forces at the interface of porous structure to the solid substrate.

An additional method that has been attempted is to drill a series of holes into a solid substrate. However, it has been found that straight holes do not stimulate adequate both growth and fusion. Furthermore, the drilling patterns required to achieve a 60% porosity create number manufacturing issues, as materials like PEEK flex during drilling, resulting in one pore breaking into a neighboring pore. Additionally, this can leave the IBF implant weak and vulnerable to failure upon implantation.

Therefore, there is a need in the art for an IBF implant and related manufacturing method that can, within a single implant, incorporate both nonporous and porous sections to provide both adequate structural support and promote optimal bone growth and fusion. Additionally, there is a need in the art for an IBF implant incorporating a purposefully engineered and osteo-conductive three-dimensional matrix that can more effectively promote bone growth and fusion by providing a pore structure with more consistent pore size and porosity. The present invention is provided to solve the problems discussed above and other problems, and to provide advantages and features not provided by currently available IBF implants. These and other features and advantages of the present invention will be explained and will become obvious to one skilled in the art through the summary of the invention that follows.

### SUMMARY OF THE INVENTION

Accordingly, embodiments of the present invention are directed to an IBF implant with a purpose built three-dimensional (“3D”) matrix that is optimized for bony ingrowth and overmolded with a material that provides structural support. Embodiments of the present invention contemplate a precisely engineered tubular network that forms a 3D matrix, which provides a porous region at any desired location within an IBF implant and with any desirable pore structure, size, or porosity. The purpose built 3D matrix is preferably made of an osteo-conductive material or coated or infused with an osteo-conductive substance.

According to an embodiment of the present invention, an interbody fusion implant comprising a three-dimensional matrix configured as a network of continuously interconnected tubes that provide a porous region within the implant for bony ingrowth, and a support material that is overmolded around the three-dimensional matrix to provide structural support for the implant.

According to an embodiment of the present invention, the IBF plant further comprises a cap sealing each open tube end of the network of continuously interconnected tubes to prevent the support material from entering the three-dimensional matrix.

According to an embodiment of the present invention, the cap is configured to be machined off to expose each the open tube end.

According to an embodiment of the present invention, the cap is bio-resorbable.

According to an embodiment of the present invention, each tube of the network of continuously interconnected tubes has a roughed interior surface.

According to an embodiment of the present invention, each tube of the network of continuously interconnected tubes is non-straight.

According to an embodiment of the present invention, each tube of the network of continuously interconnected tubes has a diameter between 0.5 mm and 1.0 mm.

According to an embodiment of the present invention, each tube of the network of continuously interconnected tubes intersects with another tube of the continuously interconnected network of tubes every 3.0 mm to 4.0 mm.

According to an embodiment of the present invention, the three-dimensional matrix is formed from an osteo-conductive material.

According to an embodiment of the present invention, the osteo-conductive material is one or more of hydroxyl apatite, bioactive glass, or titanium.

According to an embodiment of the present invention, the support material is radiolucent.

According to an embodiment of the present invention, the support material is polyether ether ketone.

According to an embodiment of the present invention, the support material is infused with an osteo-conductive material.

According to an embodiment of the present invention, the three-dimensional matrix is designed and overmolded with the support material to form rod stock that is machined into a desired implant design.

According to an embodiment of the present invention, the three-dimensional matrix is designed and overmolded to produce an application specific implant design.

According to an embodiment of the present invention, the application specific implant design is configured as a spinal implant.

According to an embodiment of the present invention, an interbody fusion implant, the implant comprising: a three-dimensional matrix configured as a network of interconnected solid branches, wherein the three-dimensional matrix is formed from a bio-resorbable material that, once implanted, dissolves to provide a porous region within the implant for bony ingrowth, and a support material that is overmolded around the three-dimensional matrix to provide structural support for the implant.

According to an embodiment of the present invention, the three-dimensional matrix is coated with an osteo-conductive material.

According to an embodiment of the present invention, the support material is polyether ether ketone.

According to an embodiment of the present invention, the support material injection molded over the three-dimensional matrix.

The foregoing summary of the present invention with the preferred embodiments should not be construed to limit the scope of the invention. It should be understood and obvious to one skilled in the art that the embodiments of the invention thus described may be further modified without departing from the spirit and scope of the invention. Other features and advantages of the invention will be apparent from the following specification taken in conjunction with the following drawings. While many materials and methods of design have been attempted, a number of drawback and limitations prevent an optimal design.

### BRIEF DESCRIPTION OF THE DRAWINGS

FIG. 1 is a zoomed-in cross-sectional view of a portion of an IBF implant with an overmolded hollow 3D matrix, in accordance with an embodiment of the present invention;

FIG. 2 is a zoomed-in cross-sectional view of a portion of an IBF implant with an overmolded hollow 3D matrix including caps for the hollow tubes, in accordance with an embodiment of the present invention;

FIG. 3 is general purpose rod stock featuring an overmolded hollow 3D matrix in accordance with an embodiment of the present invention, in accordance with an embodiment of the present invention;

FIG. 4 is a hollow 3D matrix, in accordance with an embodiment of the present invention;

FIG. 5 is an application specific IBF implant featuring an overmolded hollow 3D matrix, in accordance with an embodiment of the present invention; and

FIG. 6 is an internal view of an application specific IBF implant showing the hollow 3D matrix inside the implant, in accordance with an embodiment of the present invention.

### DETAILED DESCRIPTION

The present invention generally relates to an interbody fusion implant. Specifically, the present invention relates to an implant that incorporates a purposefully designed three-dimensional matrix that optimizes bony ingrowth, with such matrix then being overmolded with a material that provides structural support.

According to an embodiment of the present invention, an IBF implant apparatus designed with both porous and nonporous sections in a single, unified form. In a preferred embodiment, a purpose built hollow 3D matrix, which is optimum for bony ingrowth, is formed by a network of continuously interconnected tubes to provide a porous region in the implant into which the bone can grow. The 3D matrix is then overmolded with a material that provides structural support to the implant. In the preferred embodiment, the 3D matrix is made from or coated with hydroxyl apatite (“HA”) due to its osteo-conductive properties. However, any variety of osteo-conductive materials could be used including, but not limited to, various bioactive glasses and titanium. The material used to overmold the 3D matrix is preferably PEEK, but could be any biocompatible material. The use of PEEK for the overmolded portion of IBF implant is advantageous because PEEK has an elastic modulus that is similar to bone and is radiolucent making it easier to observe bone growth and fusion within and around the implant. Importantly, other materials may be incorporated into the overmolded material, including, but not limited to, HA, bioactive glasses, and any combination thereof, to further enhance the stimulation of bony ingrowth. Furthermore, the overmolded material is preferably an injection moldable material that necessarily has a melting temperature that is lower than that of the material forming the 3D matrix. One of ordinary skill in the art would appreciate that there are many other materials from which an IBF implant could be fabricated, and embodiments of the present invention are contemplated for use with any such material.

According to an embodiment of the present invention, the 3D matrix is 3D printed, thereby allowing the creation of a precise structure. In a preferred embodiment, 3D printing is capable of repeatedly creating 3D matrixes with identical specifications, including, but not limited to 3D matrixes with the same dimensions, pore structure, porosity, and pore size, with the only limitation on the implant design and size being the size of the machinery producing it. Additionally, each of these methods is capable of creating the porous structure within any section of the implant and at any depth. One of ordinary skill in the art would appreciate there are a variety of methods for implementing the 3D printing technique, and embodiments of the present invention are contemplated for use with any such methods.

According to an embodiment of the present invention, the 3D matrix is a network of continuously connected tubes. In a preferred embodiment, each of the hollow tubes has a diameter between 0.5 mm and 1.0 mm. In some embodiments, however, the tubes may be smaller than 0.5 mm or larger than 1.0 mm. In the preferred embodiment, each tube of the 3D matrix intersects with another tube of the 3D matrix every 3.0 mm to 4.0 mm. For the sake of clarity, each tube preferably proceeds uninterrupted for a length of approximately 3.0 mm to 4.0 mm before intersecting with another tube, however the interval may be longer or shorter depending upon the intended application of the IBF implant. Preferably, the tubes of the 3D matrix are not in straight lines, but have curves, twists, angles, and other changes of direction, as this has been shown to improve bone growth and fusion. However, the 3D matrix could be configured with straight line tube structures if beneficial for a particular application. Additionally, the internal surface of the tubes of the 3D matrix is preferably roughened, again to improve the growth and fusion of the bone tissue. One of ordinary skill in the art would appreciate that 3D matrix could be precisely designed with a tubular structure based upon the intended application or purpose of the implant, and embodiments of the present invention are contemplated for use with any such design.

According to an alternate embodiment of the present invention, the 3D matrix of the IBF implant is a solid matrix. In the alternate embodiment, the solid 3D matrix is formed from a bio-resorbable material and may be coated with an osteo-conductive material. The solid 3D matrix would then be overmolded with a material to provide structural support, such as PEEK. In the alternate embodiment, the solid 3D matrix would be resorbed once the IBF implant is implanted in the patient's body, thereby allowing bony ingrowth to replace the bio-resorbable material that previously formed the solid 3D matrix. In the preferred embodiment, the solid 3D matrix forms an interconnected network of solid branches that is similar to the interconnected network of hollow tubes that form the hollow 3D matrix. For example, in the preferred embodiment of the solid 3D matrix the branches have a diameter between 0.5 mm and 1.0 mm and each branch of the solid 3D matrix intersects with another branch of the solid 3D matrix every 3.0 mm to 4.0 mm.

According to an embodiment of the present invention, the 3D matrix, whether hollow or solid, is overmolded with a material that provides structural support to the IBF implant. In a preferred embodiment, the overmolded support material, which is preferably PEEK, is injection molded over the 3D matrix so that the overmolded support material will fill in all of areas around the outside of tubular structures that form the 3D matrix. Furthermore, the 3D matrix is preferably formed such that the overmolded support material can easily fill those areas between the various tubular structures without leaving any unwanted voids, thereby ensuring optimal strength and structural integrity. In embodiments that employ a hollow 3D matrix, the 3D matrix can be designed or formed with a cap that seals the open ends of each of the hollow tubes that form the hollow 3D matrix. These caps prevent any overmolded support material from entering or accidentally filling the hollow 3D matrix during the overmolding process. As the hollow 3D matrix is preferably constructed using 3D printing techniques, the caps can be seamlessly integrated with and formed on the open tube ends during the 3D printing process. When the overmolding process is complete, there are two primary methods for removing the caps to enable bone to fuse with the IBF implant. First, the caps can be machined off to expose the hollow tubes of the 3D matrix to the bone tissue. Alternatively, the caps can be made from a bio-resorbable material that is resorbed once the IBF implant is implanted in the patient.

According to an embodiment of the present invention, the hollow 3D matrix will have a varying tubular structure thereby creating a variable porosity, or amount of hollow space, within the IBF implant. Overall, the overall porosity of a given IBF implant will be determined by the amount of hollow space created by the hollow 3D matrix relative to the amount of overmolded material forming the structure of the IBF implant. In a preferred embodiment, the overall porosity of an IBF implant may be tailored based upon the application and purpose of the implant. Furthermore, an IBF implant may have multiple individual hollow 3D matrixes positioned in different portions of the IBF plant. In this way, a given IBF implant need not have a consistent porosity throughout its entirety, but instead could have a varying porosity in different portions of the implant. The customizable nature of the 3D matrix, along with the variable design and placement of a 3D matrix within an IBF implant, allows for an IBF to be adapted based upon the intended application for the implant. Notably, the overall porosity of two different IBF implants could be the same despite having vastly different design objectives. In a preferred embodiment, the overall porosity of implant is approximately 50%, but may vary within an ideal range of between 20 to 80%. As previously noted, the placement of a given hollow 3D matrix, which forms a porous region within the IBF implant, can be anywhere in the implant, including internally. Furthermore, the use of porous and nonporous regions allow for a solid, nonporous, weight-bearing section of the IBF implant to be adjacent to, and formed continuously, with a porous section that supports bone growth and fusion. One of ordinary skill in the art would appreciate the IBF implant of the present invention could be configured with any suitable porosity percentage, and embodiments of the present invention are contemplated for use with any such porosity percentage.

According to a preferred embodiment of the present invention, the IBF implant is configured with both porous and nonporous sections. In the preferred embodiment, the ratio of porous to nonporous sections, as well as the arrangement of the porous and non-porous sections, is determined by the intended application for the IBF implant. For example, an IBF implant for use in the spine have a different design than one intended for use in the hip. Additionally, while an IBF implant with both porous and nonporous sections will generally be preferred for most applications, the IBF implant could be either entirely porous or entirely nonporous. In the preferred embodiment, the nonporous sections of the IBF implant are designed and arranged to provide structure and load bearing support, while the porous sections formed by the hollow 3D matrix provide for bone growth and fusion. As an illustrative example, the nonporous sections of an IBF implant could be an I-beam, a central bar, a ring, blocks, or any combination thereof, that are formed continuously with one or more porous sections. Furthermore, the hollow 3D matrix may be covered by the nonporous sections or formed between or around the nonporous sections of the implant. Alternatively, the IBF implant may be configured with a nonporous top section that is united with a nonporous bottom section by a porous middle section. Additionally, an IBF implant may be configured with a nonporous ring that is formed continuously with a porous section on the outside and/or inside of the nonporous ring. Overall, the 3D matrix can either feature a general purpose design that can be overmolded into rod stock, which can then be machined into various implant designs, or feature a customized design for an application specific implant, which can then be overmolded to create the specific singular implant. One of ordinary skill in the art would appreciate that there are many suitable designs and arrangements for porous and nonporous sections of an IBF implant, and embodiments of the present invention are contemplated for use with any such design or arrangement.

According to an embodiment of the present invention, the IBF implant may be configured with ridged or textured areas on the outer surfaces of the implant. In a preferred embodiment, the ridged surface areas enable the implant to bite or grip onto bone neighboring the implantation area, thereby preventing unwanted migration or movement of the IBF implant. In a preferred embodiment, the ridged or textured area may be formed in any suitable configuration, including, but not limited to, corrugated patterns, diamond patterns, pyramid shapes, or any combination thereof. Additionally, in embodiments where the porous sections of the implant are on the outer surface, the porous sections could also function as the ridged or textured areas. One of ordinary skill in the art would appreciate that there are many ways to configure a ridged or textured area on an IBF implant to prevent unwanted migration and movement, and embodiments of the present invention are contemplated for use with any such configuration.

According to an embodiment of the present invention, the IBF implant may be configured with a hollow core. In a preferred embodiment, the hollow core is configured to receive an autogenous bone graft. In the preferred embodiment, the autogenous bone graft is osteo-inductive and starts the fusion process. The fusion process is further enhanced by the porous sections of the IBF implant, as the porous sections are osteo-conductive and provide a place in which bone growth and fusion more readily occurs relative to non-porous sections. One of ordinary skill in the art would appreciate that there are many suitable designs for incorporating a hollow core into an IBF implant, and embodiments of the present invention are contemplated for use with any such hollow core design.

According to an embodiment of the present invention, the IBF implant may be configured with one or more cavities in one or more walls of the implant. In some embodiments, a cavity may be a hole that goes entirely through a wall of the implant. In some embodiments, a cavity is a hollow or indentation in a wall of the implant. In the preferred embodiment, a cavity may be added to the IBF implant to adapt the implant to a certain application or to improve the performance of the implant. One of ordinary skill in the art would appreciate that there are many suitable designs and layouts for incorporating cavities into the walls of an IBF implant, and embodiments of the present invention are contemplated for use with any such cavity design and layout.

According to an embodiment of the present invention, the 3D matrix of the IBF implant is designed with a precise tubular structure based on the implants intended application. In a preferred embodiment, the 3D matrix of the IBF implant may be designed using computer-aided design (“CAD”) techniques. In the preferred embodiment, the tubular structure is configured in a CAD model that can then be used to control 3D printing manufacturing process. Furthermore, the 3D matrix created by 3D printing methods can be tailored to create specific tube sizes and structures. Finally, the use of CAD models in conjunction with 3D printing, allows for the precise reproduction of identical IBF implants. This was not possible with previous methods, such as the gas-evaporative method, which could not replicate IBF implants with identical structures.

### Exemplary Embodiments

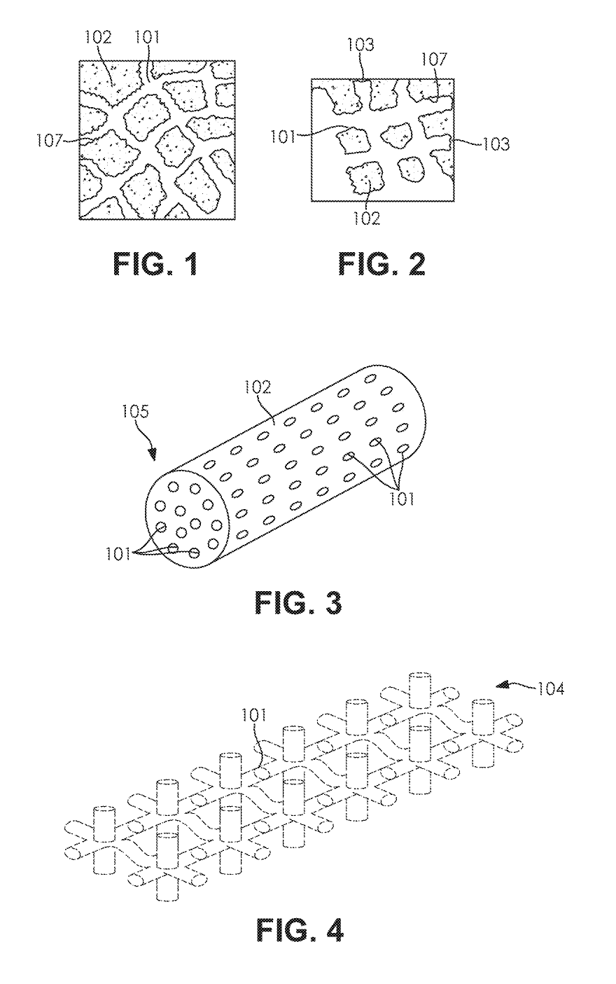

Turning now to FIGS. 1 and 2, a zoomed-in cross-sectional view of a portion of an IBF implant with an overmolded hollow 3D matrix in accordance with an embodiment of the present invention. In a preferred embodiment, the IBF implant is comprised of series of continuously interconnected tubes 101 that provide a porous region within the IBF implant that supports bony ingrowth. Preferably, the tubes 101 of the 3D matrix are curved, twisted, angled, or other have changes of direction and have a roughened interior surface 107, as these characteristics been shown improve bone growth and fusion. To give the IBF structural support, the tubes 101 are overmolded with a support material 102, such as PEEK. In the preferred embodiment, the tubes 101 of the 3D matrix are formed such that the support material 102 can fill in all of the space around the outside of the tubes 101, thereby preventing unwanted voids that can decrease the strength of the implant. In some embodiments, the ends of the tubes 101 are closed of a cap 103 that prevents support material 102 from entering the tubes 101 of the 3D matrix.

Turning now to FIG. 3, a general purpose rod stock featuring an overmolded hollow 3D matrix in accordance with an embodiment of the present invention. In some embodiments, the IBF implant may be machined into various implant designs from a general purpose rod stock 105. The general purpose rod stock 105 is preferably comprised of a hollow 3D matrix that is overmolded with a support material 102 such that the network of tubes 101 that form the hollow 3D matrix are exposed on the outer surface of rod stock 105.

Turning now to FIG. 4, an exemplary embodiment of a hollow 3D matrix in accordance with an embodiment of the present invention. In a preferred embodiment, the hollow 3D matrix 104 is a network of continuously interconnected hollow tubes 101, wherein the hollow tubes 101 twist and curve. The hollow 3D matrix 104 is designed to permit the support material to readily fill any open space around the outside of the hollow tubes 101 without leaving any pockets or voids.

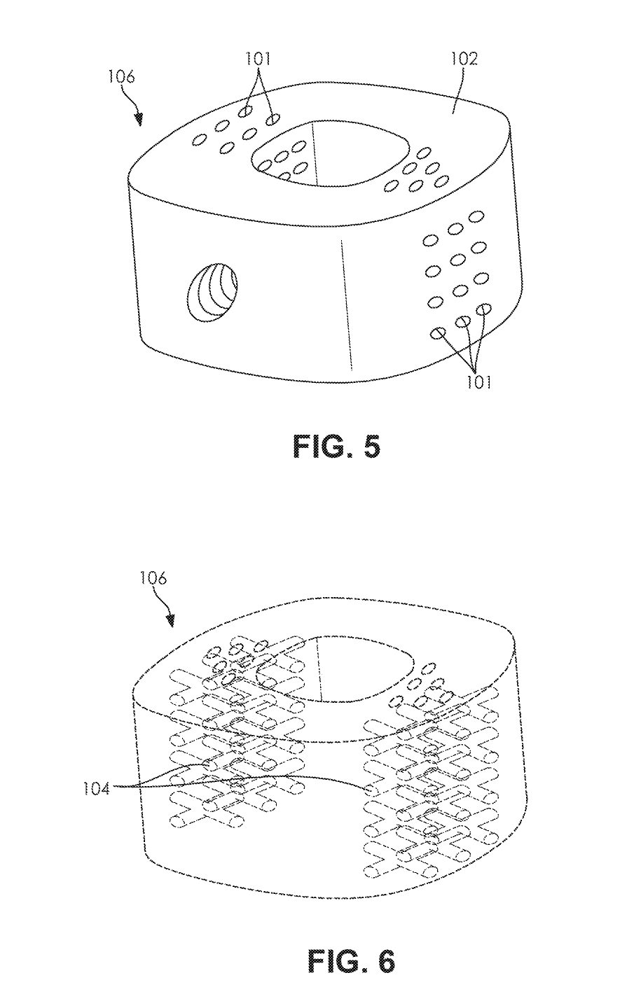

Turning now to FIG. 5, an exemplary embodiment of an application specific IBF implant featuring an overmolded hollow 3D matrix in accordance with an embodiment of the present invention. In a preferred embodiment, the application specific implant 106 is designed to meet the demands and specifications of a particular implant application, for example a spinal IBF implant. In a given application specific implant 106, the tubes 101 of the 3D matrix may only be incorporated into certain portions of the implant, leaving other portions of the application specific implant 106 solid and filled-in by the support material 102. Similarly, support material 102 may be overmolded in a precise manner to form a specific shape.

Turning now to FIG. 6, an internal view of an application specific IBF implant in accordance with an embodiment of the present invention. In a preferred embodiment, the application specific implant 106 may employ multiple, independent 3D matrixes 104 depending upon the intended application of a given implant. By using multiple 3D matrixes 104, an implant can be designed that directs and supports bony ingrowth in the particular areas of the application specific implant 106 in which such bony ingrowth is desired, while still providing solid areas within the application specific implant 106 that are configured to provide structural support.

While multiple embodiments are disclosed, still other embodiments of the present invention will become apparent to those skilled in the art from this detailed description. There may be aspects of this invention that may be practiced without the implementation of some features as they are described. It should be understood that some details have not been described in detail in order to not unnecessarily obscure focus of the invention. The invention is capable of myriad modifications in various obvious aspects, all without departing from the spirit and scope of the present invention. It should be noted that the features illustrated in the drawings are not necessarily drawn to scale, and features of one embodiment may be employed with other embodiments as the skilled artisan would recognize, even if not explicitly stated herein. Accordingly, the drawings and descriptions are to be regarded as illustrative in nature and not restrictive.

What is claimed is:

1. An interbody fusion implant, said implant comprising:
a three-dimensional matrix configured as a network of continuously interconnected tubes that provide a porous region within said implant for bony ingrowth; and
a support material that is overmolded around said three-dimensional matrix to provide structural support for said implant.

2. The implant of claim 1, further comprising a cap sealing each open tube end of said network of continuously interconnected tubes to prevent said support material from entering said three-dimensional matrix.

3. The implant of claim 2, wherein said cap is configured to be machined off to expose each said open tube end.

4. The implant of claim 2, said cap is bio-resorbable.

5. The implant of claim 1, wherein each tube of said network of continuously interconnected tubes has a roughed interior surface.

6. The implant of claim 1, wherein each tube of said network of continuously interconnected tubes is non-straight.

7. The implant of claim 1, wherein each tube of said network of continuously interconnected tubes has a diameter between 0.5 mm and 1.0 mm.

8. The implant of claim 1, wherein each tube of said network of continuously interconnected tubes intersects with another tube of said continuously interconnected network of tubes every 3.0 mm to 4.0 mm.

9. The implant of claim 1, wherein said three-dimensional matrix is formed from an osteo-conductive material.

10. The implant of claim 9, wherein said osteo-conductive material is one or more of hydroxyl apatite, bioactive glass, or titanium.

11. The implant of claim 1, wherein said support material is radiolucent.

12. The implant of claim 11, wherein said support material is polyether ether ketone.

13. The implant of claim 11, wherein said support material is infused with an osteo-conductive material.

14. The implant of claim 1, wherein said three-dimensional matrix is designed and overmolded with said support material to form rod stock that is machined into a desired implant design.

15. The implant of claim 1, wherein said three-dimensional matrix is designed and overmolded to produce an application specific implant design.

16. The implant of claim 15, wherein in said application specific implant design is configured as a spinal implant.

17. An interbody fusion implant, said implant comprising:
a three-dimensional matrix configured as a network of interconnected solid branches, wherein said three-dimensional matrix is formed from a bio-resorbable material that, once implanted, dissolves to provide a porous region within said implant for bony ingrowth; and
a support material that is overmolded around said three-dimensional matrix to provide structural support for said implant.

18. The implant of claim of claim 17, wherein said three-dimensional matrix is coated with an osteo-conductive material.

19. The implant of claim of claim 17, wherein said support material is polyether ether ketone.

20. The implant of claim of claim 17, wherein said support material injection molded over said three-dimensional matrix.
